SEQUENCE LISTING (10 sequences)

SEQ ID NO: 1 - DNA, 201 bp, Apium graveolens
  source 1..201: mol_type = genomic DNA
atatagtgaaaagaataagagggtgaggatggggctgtagcctgtagcacaaacgcacacaggaggtagcttagctctgctggaccgcttcatgcctctcttttttatgccaaaagtacaaaccaaaaaatagtaaacaaagcaattgtattttcgaaaacataaaacattagccgaaaggaactaaaacaattgagcatg

SEQ ID NO: 2 - DNA, 201 bp, Apium graveolens
  source 1..201: mol_type = genomic DNA
atgcatatcacgtaacacataatcatgttattcatatatcacgtaatcacatattccatgtaacayataagtcaggttgtcaaaayataggttttaggacgttcagaattgaaatcgggtcaataaccgggtttatcgatcagctaycgactcagtataactcacaaatcaaatgacattgctatacaaaaggaattaggt

SEQ ID NO: 3 - DNA, 52 bp, synthetic construct
  source 1..52: mol_type = other DNA
gaaggtgaccaagttcatgctattttttggtttgtacttttggcataaaaaa

SEQ ID NO: 4 - DNA, 54 bp, synthetic construct
  source 1..54: mol_type = other DNA
gaaggtcggagtcaacggattctattttttggtttgtacttttggcataaaaat

SEQ ID NO: 5 - DNA, 22 bp, synthetic construct
  source 1..22: mol_type = other DNA
tgctggaccgcttcatgcctct

SEQ ID NO: 6 - DNA, 48 bp, synthetic construct
  source 1..48: mol_type = other DNA
gaaggtgaccaagttcatgctgttattgacccgatttcaattctgaac

SEQ ID NO: 7 - DNA, 49 bp, synthetic construct
  source 1..49: mol_type = other DNA
gaaggtcggagtcaacggattggttattgacccgatttcaattctgaat

SEQ ID NO: 8 - DNA, 30 bp, synthetic construct
  source 1..30: mol_type = other DNA
agtcaggttgtcaaaayataggttttagga

SEQ ID NO: 9 - DNA, 201 bp, Apium graveolens
  source 1..201: mol_type = genomic DNA
atatagtgaaaagaataagagggtgaggatggggctgtagcctgtagcacaaacgcacacaggaggtagcttagctctgctggaccgcttcatgcctctcatttttatgccaaaagtacaaaccaaaaaatagtaaacaaagcaattgtattttcgaaaacataaaacattagccgaaaggaactaaaacaattgagcatg

SEQ ID NO: 10 - DNA, 201 bp, Apium graveolens
  source 1..201: mol_type = genomic DNA
atgcatatcacgtaacacataatcatgttattcatatatcacgtaatcacatattccatgtaacayataagtcaggttgtcaaaayataggttttaggacattcagaattgaaatcgggtcaataaccgggtttatcgatcagctaycgactcagtataactcacaaatcaaatgacattgctatacaaaaggaattaggt

(19) United States
(12) Patent Application Publication
(10) Pub. No.: US 2025/0380657 A1
(43) Pub. Date: Dec. 18, 2025

(54) REDUCED FOLIAGE CELERY

(71) Applicant: RIJK ZWAAN ZAADTEELT EN ZAADHANDEL B.V., De Lier (NL)

(72) Inventors: Reinier Hendrik Marie DENEER, De Lier (NL); Evert Willem GUTTELING, De Lier (NL); Pieternella Jozina DE PREE, De Lier (NL)

(21) Appl. No.: 19/285,719

(22) Filed: Jul. 30, 2025

Related U.S. Application Data
(63) Continuation-in-part of application No. PCT/EP2025/064980, filed on May 30, 2025.
(63) Continuation-in-part of application No. 18/054,630, filed on Nov. 11, 2022, now Pat. No. 12,433,217.
Application No. 18/054,630 is a continuation of application No. 13/336,477, filed on Dec. 23, 2011, now Pat. No. 11,690,337.

(30) Foreign Application Priority Data
Dec. 23, 2010 (NL) 2005919
May 31, 2024 (WO) PCT/EP2024/065096
Dec. 19, 2024 (WO) PCT/EP2024/087507

(51) Int. Cl. A01H 6/06; A01H 5/04

(52) U.S. Cl. CPC A01H 6/064; A01H 5/04

(57) ABSTRACT

Celery plant (Apium graveolens L. dulce) with QTL on chromosome 8 between SEQ ID No. 1 and SEQ ID No. 2, which when homozygously present is responsible for the presence of two internodes on average per leaf at the harvesting stage (Reduced foliage or “Rf”-type ”). The QTL can be genetically linked to at least one SNP present in SEQ ID No. 1 and/or SEQ ID No. 2. Celery seed deposited as NCIMB 44381 or NCIMB 41513 has the QTL. Celery plant having the QTL or genetic determinant having the QTL leads to the Rf-type (compared to celery not carrying the genetic determinant or QTL). The determinant or QTL is obtainable by introgression from a plant from NCIMB 41513 or 44381. Crossing celery from NCIMB 44381 or NCIMB 41513 with wildtype celery results in segregated F2 progeny population wherein Rf-type is observed in a monogenic recessive fashion. Methods for producing or growing the celery plant, and seeds, propagation material, progeny, and food products of, from the celery plant also disclosed.

### RELATED APPLICATIONS/INCORPORATION BY REFERENCE

This application is a continuation-in-part of PCT/EP2025/064980, filed 30 May 2025 and designating the United States, and claiming Paris Convention priority to PCT/EP2024/065096, filed 31 May 2024 and PCT/EP2024/087507, filed 19 Dec. 2024.

This application is also a continuation-in-part of U.S. application Ser. No. 18/054,630, filed Nov. 11, 2022 (published as US 20230172134A1, and now allowed), as a continuation of U.S. application Ser. No. 13/336,477, filed Dec. 23, 2011, with a claim of Paris Convention priority to Dutch (the Netherlands) Application 2005919, filed Dec. 23, 2010 (U.S. Ser. No. 13/336,477 published as US 20120164304A1 and now U.S. Pat. No. 11,690,337).

Reference is made to U.S. Pat. No. 12,193,381, issued from U.S. application Ser. No. 18/054,639, filed Nov. 11, 2022 as a continuation of U.S. application Ser. No. 13/336,477, filed Dec. 23, 2011 (now U.S. Pat. No. 11,690,337). Reference is also made to US 20230073853A1, the publication of U.S. application Ser. No. 18/054,639.

Each and all of: PCT/EP2025/064980, filed 30 May 2025; PCT/EP2024/065096, filed 31 May 2024; PCT/EP2024/087507, filed 19 Dec. 2024; U.S. application Ser. No. 18/054,630, filed Nov. 11, 2022; US 20230172134A1, U.S. application Ser. No. 13/336,477, filed Dec. 23, 2011; US 20120164304A1; U.S. Pat. No. 11,690,337; U.S. Pat. No. 12,193,381; U.S. application Ser. No. 18/054,639, filed Nov. 11, 2022; and US 20230073853A1; and Dutch (the Netherlands) Application 2005919, filed Dec. 23, 2010, are hereby incorporated herein by reference as if set out in full herein. In addition, the entire prosecution history of each of U.S. application Ser. No. 13/336,477, filed Dec. 23, 2011, U.S. application Ser. No. 18/054,639, filed Nov. 11, 2022, and U.S. application Ser. No. 18/054,630, filed Nov. 11, 2022, are hereby incorporated herein by reference as if set out in full herein.

Moreover, the foregoing applications, patent publications and patents, and all documents cited therein or during their prosecution and their entire prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE STATEMENT

The instant application contains a Sequence Listing which has been submitted electronically and is hereby incorporated by reference in its entirety. Said XML copy was created May 27, 2025 and amended Aug. 5, 2025, is named Y7954_03063ReplacementSL.xml and is 10,713 bytes in size.

### FIELD OF THE INVENTION

The present invention relates to a new type of celery (Apium graveolens L. dulce). The invention further relates to a method for producing such Apium graveolens L. dulce plant and to methods for identification and selection of such a celery plant. The invention also relates to progeny and seeds of such a celery plant, to propagation material suitable for producing such a celery plant, and to a food product comprising such a celery plant or part thereof. The invention further relates to a cell or a tissue culture that is produced from, or can be regenerated into such a celery plant. The invention also relates to markers for identification in such celery plant and to use of said markers.

### BACKGROUND OF THE INVENTION

Apium graveolens is a plant species belonging to the Apiaceae family, which comprises two important vegetable crops, namely celery and celeriac. In Apium graveolens L. dulce (celery, also known as stalk celery), the plant part that is typically harvested for consumption are the first internodes of the leaves.

A single celery plant typically represents a large amount of biomass, and in particular market segments an entire celery plant is often too large for the consumer to store conveniently. Moreover, celery is a vegetable that has a relatively large amount of unusable parts. On average 50% of the leaves is not sold, but cut off as waste. Typically, celery plants comprise multiple leaves. The leaves of celery are compound leaves that are composed of a petiole or leaf stem (which is the first internode of the celery leaf, extending from the leaf base up to the first node) and a fully subdivided leaf blade. The leaf blade typically consists of several internodes, each with a pair of leaflets, and it ends in a terminal leaflet that sprouts from the uppermost node, along with two regular leaflets. FIG. 1 illustrates that for industrial processing a celery plant is usually cut off just below the first pair of leaflets growing from the first node. The first internode is the marketable section of the celery plant that is processed and consumed, while everything above the first internode is usually discarded as waste. The first internode or petiole is also known as the stalk, and after removal of the leaflets it is usually called “stick”. The petiole carries a leaf blade, and a petiole and its leaf blade together form a leaf of the celery plant.

### SUMMARY OF THE INVENTION

It is an object of the present invention to provide a new type of celery with a leaf architecture that allows for an optimized use of its biomass and a minimal amount of waste, and that is also better suited for machine-harvesting and for machine-processing than wildtype celery.

In the research leading to the present invention, it was found that the homozygous presence of a specific Quantitative Trait Locus (QTL) on chromosome 8 of the celery genome causes a leaf architecture that is very different from the leaf architecture of a wildtype celery plant (Example 1). This research further revealed that said QTL is located between marker RF1 (SEQ ID No. 1) and marker RF2 (SEQ ID No. 2). The QTL is inherited in a recessive fashion. Embodiments of the invention may be summarized by the following numbered paragraphs:
1. A cultivated Apium graveolens L. dulce plant comprising a QTL on chromosome 8 which is located between SEQ ID No. 1 and SEQ ID No. 2, which QTL when homozygously present is responsible for the presence of two internodes on average per leaf at the harvesting stage.
2. A cultivated Apium graveolens L. dulce plant as in paragraph 1, wherein the average number of internodes per leaf at the harvesting stage is two.
3. A cultivated Apium graveolens L. dulce plant as in paragraph 1 or 2, wherein the QTL on chromosome 8 is genetically linked to at least one of the SNPs as presented in SEQ ID No. 1 and SEQ ID No. 2.
4. A cultivated Apium graveolens L. dulce plant as in paragraph 1, wherein the QTL is as comprised in the genome of an Apium graveolens L. dulce plant representative seed of which was deposited with the NCIMB under deposit number NCIMB 44381 or NCIMB 41513.
5. A cultivated Apium graveolens L. dulce plant as in paragraph 1, wherein the QTL is introgressed from a plant grown from seed deposited with the NCIMB under NCIMB 44381 or NCIMB 41513, or from a progeny plant thereof that has retained the QTL.
6. An Apium graveolens L. dulce seed of, from, or that produces the cultivated Apium graveolens L. dulce plant as in paragraph 1 and comprises the QTL on chromosome 8.
7. An Apium graveolens L. dulce seed comprising a QTL on chromosome 8 which is located between SEQ ID No. 1 and SEQ ID No. 2, which QTL when homozygously present is responsible for the presence of two internodes on average per leaf at the harvesting stage of a cultivated Apium graveolens L. dulce plant grown from the seed.
8. Propagation material from, or capable of developing into and/or derived from an Apium graveolens L. dulce plant according to paragraph 1, wherein the propagation material is suitable for sexual reproduction, and comprises a microspore, pollen, an ovary, an ovule, an embryo sac, or an egg cell; or wherein the propagation material is suitable for vegetative reproduction, and comprises a cutting, a root, a stem, a cell, or a protoplast; or wherein the propagation material is suitable for tissue culture of regenerable cells, and comprises a leaf, pollen, an embryo, a cotyledon, a hypocotyl, a meristematic cell, a root, a root tip, an anther, a flower, a seed, or a stem; and wherein the plant produced from the propagation material comprises the QTL on chromosome 8.
9. A method for producing a cultivated Apium graveolens L. dulce plant having two internodes on average per leaf at the harvesting stage, said method comprising:
a) crossing a first Apium graveolens L. dulce plant with a second Apium graveolens L. dulce plant to obtain a first generation population, wherein the first Apium graveolens L. dulce plant is an Apium graveolens L. dulce plant according to paragraph 1;
b) performing one or more rounds of selfing and/or crossing of the plant resulting from the cross to obtain a further generation population;
c) phenotypically selecting from among the plants resulting from the further generation population of step b) a plant that has two internodes on average per leaf at the harvesting stage.
   10. The method as in paragraph 9, wherein the first plant is a plant grown from seed deposited under NCIMB accession number NCIMB 44381 or NCIMB 41513, or a progeny plant thereof that has retained the QTL on chromosome 8.
   11. A method for producing Apium graveolens L. dulce seed comprising the QTL on chromosome 8 comprising producing a cultivated Apium graveolens L. dulce plant according to paragraph 9, and harvesting seed therefrom.
   12. A method for producing Apium graveolens L. dulce seed comprising the QTL on chromosome 8 comprising producing a cultivated Apium graveolens L. dulce plant according to claim 10, and harvesting seed therefrom.
   13. A method for producing cultivated hybrid Apium graveolens L. dulce seed comprising crossing a first Apium graveolens L. dulce parent plant with a second Apium graveolens L. dulce parent plant and harvesting the resultant hybrid seed, wherein the first parent plant and the second parent plant are plants comprising homozygously a QTL on chromosome 8 which is located between SEQ ID No. 1 and SEQ ID No. 2, which QTL when homozygously present in an Apium graveolens L. dulce plant is responsible for the presence of two internodes on average per leaf at the harvesting stage.
   14. The cultivated hybrid Apium graveolens L. dulce seed produced by the method of paragraph 11.
   15. The cultivated hybrid Apium graveolens L. dulce seed produced by the method of paragraph 12.
   16. The cultivated hybrid Apium graveolens L. dulce seed produced by the method of paragraph 13.
   17. A method for growing a cultivated Apium graveolens L. dulce plant with two internodes on average per leaf at the harvesting stage, which method comprises the step of germinating a seed according to paragraph 14.
   18. A method for growing a cultivated Apium graveolens L. dulce plant with two internodes on average per leaf at the harvesting stage, which method comprises the step of germinating a seed according to paragraph 15.
   19. A method for growing a cultivated Apium graveolens L. dulce plant with two internodes on average per leaf at the harvesting stage, which method comprises the step of germinating a seed according to paragraph 16.
   20. The Apium graveolens L. dulce seed of paragraph 6, that produces the plant of cultivated Apium graveolens L. dulce plant as in paragraph 1 and comprises the QTL on chromosome 8, wherein said seed is seed deposited under NCIMB accession number NCIMB 44381 or NCIMB 41513.
   21. A cultivated Apium graveolens L. dulce plant comprising homozygously a QTL on chromosome 8 which is located between SEQ ID No. 1 and SEQ ID No. 2, whereby the Apium graveolens L. dulce plant exhibits the trait of two internodes on average per leaf at the harvesting stage, i.e., wherein the Apium graveolens L. dulce plant is Reduced foliage (RF) Celery.
   22. The cultivated Apium graveolens L. dulce plant of paragraph 21, wherein the plant is grown from seed deposited under NCIMB accession number NCIMB 44381 or NCIMB 41513.

The invention thus provides a celery plant of the species Apium graveolens L. dulce with a “reduced foliage” (Rf) architecture. Wildtype celery plants typically have three, four or even five internodes plus a terminal leaflet, whereby the largest part of the second internode and all internodes above that point (and the terminal leaflet) are usually cut off and discarded as waste after harvest. The Reduced foliage (Rf) celery type has only two internodes on average per leaf at the harvesting stage (FIG. 3). Typically, its second internode is much shorter than that in wildtype celery plants. The first internode (the petiole, stalk or stick) is equally long or longer than in wildtype celery plants (FIG. 4). As a result of this, Rf celery plants are generally shorter than wildtype celery plants when grown in identical conditions, but they have a much higher proportion of commercially usable biomass (Example 2).

The final number of internodes and nodes in a celery leaf is already established at an early developmental stage. Therefore, the Rf-type can already be recognized long before the harvesting stage, in young, immature leaves. It is therefore not necessary to wait until the plant is ready for harvest to assess the presence or absence of the Rf-type in any given celery plant.

The term “two internodes on average per leaf” is intended to mean that all leaves of an Rf-type celery plant of the invention typically comprise two internodes. However, it may happen that an Rf-type plant has one or more leaves with only one and/or three internodes. When the total number of internodes on all leaves of a single celery plant is counted, and this number is divided by the number of leaves on that celery plant, this will result in an average number of internodes per leaf that is in any case significantly lower than three, and typically about two. The average number of internodes per leaf within a plant of the invention is thus about 1.5, about 1.6, about 1.7, about 1.8, about 1.9, about 2.0, about 2.1, about 2.2, about 2.3, about 2.4, or about 2.5, but it is preferably about 2.0. In wildtype celery plants this average number of internodes per leaf within a plant is always about three or more (FIG. 3).

In particular, the invention relates to a cultivated celery plant (Apium graveolens L. dulce) comprising a QTL on chromosome 8 which is located between marker RFI (SEQ ID No. 1) and marker RF2 (SEQ ID No. 2), which QTL when homozygously present is responsible for the presence of two internodes on average per leaf at the harvesting stage.

“Harvesting stage” is intended to mean the stage in the celery plant's life cycle wherein its stalks are physiologically mature and ready for consumption. Typically, this is about four to six months after sowing the seeds, but this may depend on the environmental conditions, the plant's genotype and the consumer's preference. The person skilled in the art of celery breeding and/or celery growing knows best when his particular celery crop is at the optimal stage for harvesting, processing and consumption.

In one embodiment, the cultivated celery plant comprises a QTL on chromosome 8 that is genetically linked to at least one of the markers comprising a SNP as presented in the markers RFI (SEQ ID No. 1) and marker RF2 (SEQ ID No. 2).

As used herein, a marker is genetically linked to, and can therefore be used for the identification of a QTL of the invention, when the marker and the Rf-type phenotype co-segregate in a segregating population resulting from a cross between a plant comprising a QTL of the invention and a plant lacking the QTL. A marker genetically linked to a QTL can be used for identification of that QTL because a linked marker is present in said QTL.

It is also an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph), such that Applicants reserve the right and hereby disclose a disclaimer of any previously described product, process of making the product, or method of using the product. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### DETAILED DESCRIPTION

Table 1 provides the sequences of the SEQ ID Nos. that can be used as markers, or that can be used to develop markers, to identify the presence of the QTL of the invention in a celery plant. As used herein, the “SNP present in” or the “SNP presented in” a certain SEQ ID No. is the nucleotide of the SNP within the sequence that is indicative of the Rf-type. Markers RF1 (SEQ ID No. 1) and marker RF2 (SEQ ID No. 2) delimit the QTL region. Table 1 also lists the position of the SNP in each sequence, the derived (mutant) allele that is linked to the Rf trait, and the wildtype allele for each SNP, as well as the physical position of each SNP on the public Api-gra_Ventura_v1 genome assembly (PMID 33095976; Song et al., 2020, Plant Biotechnol. 19:731-744). With reference to this genome assembly, the QTL region of the invention is located on chromosome 8 between positions 229,801,360and 237,176,427. The genetic distance between the two flanking markers (RF1 and RF2) is about 0.1 cM (Example 1).

TABLE 1
Molecular markers delimiting the QTL region (RF1 and RF2),
and markers genetically linked to the Rf trait in celery.

| | | | | Nucleotide | | |
|---|---|---|---|---|---|---|
| | | | | of the | | |
| | | | | SNP in | | |
| | | | | the SEQ | | Physical |
| | | | | ID No., | | position |
| | | | | to be | | of the SNP |
| | | | Position | used as | | on the public |
| | | | of the | marker | Nucleotide | Api- |
| | | | SNP in | of the | of the | gra_Ventura_v1 |
| Marker | | Chrom. | the SEQ | invention | SNP in the | genome map |
| name | SEQ ID No. | number | ID No. | (Rf-type) | wildtype | (in bp) |
| RF1 | SEQ ID | 8 | 101 | A | T | 229,801,360 |
| | No. 1 | | | | | |
| RF2 | SEQ ID | 8 | 101 | A | G | 237,176,427 |
| | No. 2 | | | | | |

In a further embodiment, the QTL of the invention is as comprised in the genome of a celery plant representative seed of which was deposited with the NCIMB under deposit number NCIMB 44381 or NCIMB 41513. Suitably, said QTL has been introgressed into the cultivated celery plant directly from NCIMB 44381 or NCIMB 41513, or from a progeny plant thereof that has retained the QTL.

“Introgression” as used herein is intended to mean introduction of a trait into a plant not carrying the trait by means of crossing and selection in the first generation in which the trait becomes phenotypically visible, or in any later generation, or in the F1 generation, or any further generation, if molecular markers are used for detecting the trait. For a dominant trait the trait becomes visible in the F1 generation of the cross between a plant with the trait and a plant without the trait. For a recessive trait this is suitably the F2 generation.

The invention also relates to progeny of a plant, a cell, a tissue, or a seed of a cultivated celery plant of the invention, which progeny comprises on chromosome 8 the QTL region of the invention as defined herein. Such progeny can in itself be a plant, a cutting, a seed, a cell, or a tissue.

As used herein, “progeny” is intended to mean the first and all further descendants, such as an F1, F2, or further generation, from a cross with a plant of the invention, wherein a cross comprises a cross with itself or a cross with another plant, and wherein a descendant that is determined to be progeny comprises the QTL of the invention as defined herein, that when present in a homozygous state is responsible for the presence of two internodes on average per leaf at the harvesting stage. The plant of the invention that is used in this cross is optionally a plant grown from seed of deposit NCIMB 44381, or from progeny seed thereof which is a direct or further descendant through crossing a plant grown from the deposited seed with itself or with another plant for one or more subsequent generations, wherein the progeny seed has retained the QTL of the invention.

Progeny also encompasses a cultivated celery plant that carries the QTL of the invention and has leaves with two internodes on average per leaf at the harvesting stage, and that is obtained from the plant, or progeny of a plant, of the invention by vegetative propagation or another form of multiplication.

The invention also relates to a celery seed comprising the QTL of the invention in its genome, wherein a plant grown from the seed is a celery plant with leaves with two internodes on average per leaf at the harvesting stage.

The current invention also relates to propagation material capable of developing into and/or being derived from a celery plant comprising the QTL of the invention in its genome. Preferably, the propagation material is suitable for sexual reproduction, and it is in particular selected from the group comprising a microspore, pollen, an ovary, an ovule, an embryo sac, and an egg cell; or it is suitable for vegetative reproduction, and is in particular selected from the group comprising a cutting, a root, a stem, a cell, and a protoplast; or is suitable for tissue culture of regenerable cells, and is in particular selected from the group comprising a leaf, pollen, an embryo, a cotyledon, a hypocotyl, a meristematic cell, a root, a root tip, an anther, a flower, a seed, and a stem; wherein the propagation material and the plant produced from the propagation material comprises in its genome the QTL of the invention that, when homozygously present, is responsible for the presence of two internodes on average per leaf at the harvesting stage.

In one embodiment, said propagation material is derived from a celery plant, representative seed of which was deposited with the NCIMB under deposit number NCIMB 44381 or NCIMB 41513, or from a progeny plant thereof.

The invention further relates to a cell comprising the QTL of the invention as defined herein. A cell of the invention can be obtained from, or be present in, a plant of the invention. Such a cell may either be in isolated form, or a part of the complete plant, or from a part thereof, and still constitutes a cell of the invention because such a cell comprises the genetic information that determines the Rf trait as described herein. Each cell of a plant of the invention carries the QTL of the invention, and thereby the genetic information that leads to the presence of two internodes on average per leaf at the harvesting stage. A cell of the invention may also be a regenerable cell that can regenerate into a new plant of the invention. The presence of the genetic information in this context is the presence of the QTL of the invention, wherein the QTL is as defined herein.

The invention also relates to a food product comprising a celery plant of the invention, or part thereof. In particular, the invention relates to a food product comprising the stalks or sticks of such a celery plant, and to a packaging comprising such a food product.

The present invention further relates to a method for identifying a celery plant carrying the genetic trait of developing leaves comprising two internodes on average per leaf at the harvesting stage, wherein the method comprises screening a celery plant population for the presence of the QTL of the invention, and identifying a celery plant that comprises at least one of the SNP as presented in any one of the markers RFI (SEQ ID No. 1) and RF2 (SEQ ID No. 2) as a celery plant of the invention. Optionally, said method may also comprise the step of phenotypically screening for the presence of the Rf trait of the invention by counting the average number of internodes per leaf at the harvesting stage, or at an earlier developmental stage in which the difference between wildtype and Rf-type leaf architecture is already visible.

The invention also relates to a marker for the identification of the QTL on chromosome 8 in a celery plant, which marker is any one of a group comprising the SNPs presented in markers RF1 (SEQ ID No. 1) and RF2 (SEQ ID No. 2). The SNPs presented in markers RF1 (SEQ ID No. 1) and RF2 (SEQ ID No. 2) are genetically linked to said QTL on chromosome 8 and to the Rf-trait in celery. The markers can be used to screen for the presence of the QTL in cultivated plants, such as celery varieties, but also in wild celery plants and accessions. The SNPs presented in markers RF1 (SEQ ID No. 1) and RF2 (SEQ ID No. 2) are genetically linked to the Rf-type phenotype of plants grown from seed deposit NCIMB 44381 or NCIMB 41513.

This invention further relates to the use of a marker for the identification of a celery plant with two internodes on average per leaf at the harvesting stage. The markers are in particular useful for screening plants in an early developmental stage in which the phenotype is not yet visible.

This invention also relates to a method for selecting a celery plant carrying the trait of developing two internodes on average per leaf at the harvesting stage, comprising identifying the presence of the QTL on chromosome 8 of the invention, and selecting a plant that comprises said QTL in a homozygous state. Suitably, identifying the presence of the QTL on chromosome 8 is done by using a marker comprising a SNP presented in any one of SEQ ID Nos. 1 and 2 for the identification of said QTL.

A marker can be defined as a reference sequence that comprises the modification(s) that can be detected using any suitable method known. The term “marker”, “molecular marker”, “genetic marker” or “DNA marker” refers to a feature of an organism's genome (e.g. a nucleotide or a polynucleotide sequence that is present in an organism's genome) that is associated with one or more loci of interest. In some embodiments, a genetic marker is polymorphic in a population of interest. Genetic markers include, for example, single nucleotide polymorphisms (SNPs), indels (i.e. insertions/deletions), simple sequence repeats (SSRs), restriction fragment length polymorphisms (RFLPs), random amplified polymorphic DNAs (RAPDs), cleaved amplified polymorphic sequence (CAPS) markers, Diversity Arrays Technology (DArT) markers, and amplified fragment length polymorphisms (AFLPs), among many other examples. Genetic markers can, for example, be used to locate genetic loci containing alleles on a chromosome that contribute to variability of phenotypic traits. The term “marker” or “genetic marker” can also refer to a polynucleotide sequence complementary to a genomic sequence, such as a sequence of a nucleic acid used as a probe. The term “marker” then refers to a physical entity that can be used in molecular biological techniques for detecting the mutation.

In the context of the present invention, a marker allows the unambiguous detection of the QTL region that is genetically linked to the Rf-trait in celery, and the selection of celery plants that harbor in their genome the Rf-trait at any stage of their life cycle, even when the plants are only in the seedling stage. Marker-assisted breeding and selection greatly increases the speed with which a trait can be introduced into different genetic backgrounds, and with which it can be commercialized.

Methods for detecting markers and specific alleles are abundantly known in the field. In general, these methods allow to distinguish between two different alleles of a marker, on a specific chromosome. Detection of a polymorphism can be achieved by electrophoretic techniques, but the widespread availability of DNA sequencing often makes it easier to simply sequence amplified products directly. Once the polymorphic sequence difference is known, rapid assays for the detection of a polymorphism can be designed for progeny testing, generally involving some version of PCR amplification of specific alleles.

In particular examples, PCR detection and quantification is carried out using two labeled fluorogenic oligonucleotide forward primers and an unlabeled common reverse primer, for example, KASP™ (KBiosciences). Table 2 provides a list of KASP primers that are suitable for the identification of flanking markers delimiting the QTL region, and for the identification of markers genetically linked to the Rf trait in celery. The person skilled in the art of molecular biology may of course also use other types of markers, and the primers listed here are merely illustrative and not limiting in any way.

The presence or absence of marker RFI in the genome of a celery plant can be investigated in a KASP™ (KBiosciences) assay by using the primers listed in SEQ ID No. 3, SEQ ID No. 4 and SEQ ID No. 5. For investigating the presence or absence of marker RF2 the primers listed in SEQ ID No. 6, SEQ ID No. 7 and SEQ ID No. 8 can be used.

TABLE 2
KASP primers suitable for the identification of flanking markers
delimiting the QTL region, and for the identification of
markers genetically linked to the Rf trait in celery.

| | | | | SEQ ID No. |
|---|---|---|---|---|
| Primer | SEQ | Primer | Dye in the | targeted with |
| name | ID No. | description | KASP assay | this primer |
| RF1_ALT | SEQ ID | Forward, Wt | FAM | SEQ ID |
| | No. 3 | | | No. 1 |
| RF1_ALA | SEQ ID | Forward, Rf | VIC | SEQ ID |
| | No. 4 | | | No. 1 |
| RF1_C1 | SEQ ID | Reverse | None | SEQ ID |
| | No. 5 | | | No. 1 |
| RF2_ALT | SEQ ID | Forward, Wt | FAM | SEQ ID |
| | No. 6 | | | No. 2 |
| RF2_ALC | SEQ ID | Forward, Rf | VIC | SEQ ID |
| | No. 7 | | | No. 2 |
| RF2_C1 | SEQ ID | Reverse | None | SEQ ID |
| | No. 8 | | | No. 2 |

This invention further relates to a method for producing a cultivated celery plant with two internodes on average per leaf at the harvesting stage, said method comprising:
a) crossing a plant according to the invention with another plant to obtain a first generation population;
b) performing one or more rounds of selfing and/or crossing of the plant resulting from the cross to obtain a further generation population;
c) selecting from among the plants resulting from the further generation population of step b) a plant that homozygously comprises the QTL on chromosome 8 of the invention, which plant has the trait of developing two internodes on average per leaf at the harvesting stage (Example 3).

In one embodiment, selecting a plant comprising the QTL on chromosome 8 is done by using a molecular marker genetically linked to the QTL,preferably a marker comprising a SNP presented in any one of SEQ ID Nos. 1 and 2 for the identification of said QTL. In another embodiment, a plant with two internodes on average per leaf at the harvesting stage is phenotypically selected, in particular by determining the average number of internodes per leaf at the harvesting stage, or at an earlier developmental stage in which the difference between wildtype and Rf-type leaf architecture is already visible.

This invention also relates to a method for producing a cultivated celery plant with two internodes on average per leaf at the harvesting stage, said method comprising:
a) crossing a plant grown from seed deposited under NCIMB accession number NCIMB 44381, or a progeny plant thereof that has retained the QTL on chromosome 8, with another plant to obtain a first generation population;
b) performing one or more rounds of selfing and/or crossing of the plant resulting from the cross to obtain a further generation population;
c) selecting from among the plants resulting from the further generation population of step b) a plant that homozygously comprises the QTL on chromosome 8 of the invention, which plant has two internodes on average per leaf at the harvesting stage.

This invention also relates to a method for the production of cultivated hybrid celery seed comprising crossing a first parent plant with a second parent plant and harvesting the resultant hybrid seed, wherein the first parent plant and the second parent plant are plants comprising the QTL on chromosome 8 according to the invention in a homozygous state, wherein said QTL is responsible for the presence of two internodes on average per leaf at the harvesting stage in the hybrid plant that is grown from the seed. This invention also relates to the cultivated hybrid celery seed produced by this method.

The invention further relates to a method for the production of a celery plant comprising the QTL of the invention, by using tissue culture or by using vegetative propagation.

The invention further provides a method for the production of a celery plant comprising the QTL of the invention by using a doubled haploid generation technique to generate a doubled haploid line that is completely homozygous, and therefore homozygously comprises the QTL of the invention, and that has leaves with two internodes on average per leaf.

The invention further relates to a method for the production of a celery plant comprising the QTL of the invention, wherein the presence of said QTL leads to leaves with two internodes on average per leaf at the harvesting stage, which method comprises growing a seed comprising said QTL into the said plant.

The invention also relates to a method of growing a celery plant of the Rf-type and/or a celery plant comprising a QTL on chromosome 8 which is located between marker RF1 (SEQ ID No. 1) and marker RF2 (SEQ ID No. 2), comprising the step of germinating a celery seed of the invention under suitable conditions into a celery plant of the invention. Suitably, the QTL in said celery seed and celery plant is as present in the genome of a celery plant, representative seed of which was deposited with the NCIMB under deposit number NCIMB 44381 or NCIMB 41513. In one embodiment, said QTL is introgressed from NCIMB 44381 or NCIMB 41513 or from a progeny plant thereof.

This invention also relates to a QTL on chromosome 8 of the celery genome, which QTL when homozygously present is responsible for the presence of two internodes on average per leaf at the harvesting stage, and which QTL comprises a nucleotide sequence flanked by SEQ ID Nos. 1 and 2.

### DEPOSIT

Seeds of Apium graveolens L. dulce homozygously comprising the quantitative trait locus (QTL) on chromosome 8 conferring the “reduced foliage” trait of the invention, were deposited with NCIMB Ltd, Wellheads Place, Dyce, Aberdeen, AB21 7GB Scotland on 12 Apr. 2024 under deposit accession number NCIMB 44381.

The Deposit with the NCIMB, under deposit accession number NCIMB 44381 was made and accepted pursuant to the terms of the Budapest Treaty. The deposit will be irrevocably and without restriction or condition released to the public upon the issuance of a patent and for the enforceable life of the patent. Upon issuance of a patent, all restrictions upon the deposit will be removed, and the deposit is intended to meet the requirements of 37 CFR §§ 1.801-1.809. The deposit will be maintained in the depository for a period of 30years, or 5 years after the last request, or for the effective life of the patent, whichever is longer, and will be replaced if necessary during that period.

Seeds of Apium graveolens 07.8733 that comprise the genetic determinant of the invention which leads to short leaf blades and/or a more uniform distribution of stem lengths as discussed in applications and patents incorporated herein by reference (e.g., U.S. application Ser. No. 18/054,630, filed Nov. 11, 2022, U.S. application Ser. No. 18/054,639, filed Nov. 11, 2022, U.S. application Ser. No. 13/336,477, filed Dec. 23, 2011, U.S. Pat. No. 11,690,337 and U.S. Pat. No. 12,193,381) were deposited with NCIMB Ltd, Ferguson Building, Craibstone 5 Estate, Bucksburn, Aberdeen AB21 9YA Scotland, UK on 22 Oct. 2007 under deposit accession number NCIMB 41513.

The Deposit with the NCIMB, under deposit accession number NCIMB 41513 was made and accepted pursuant to the terms of the Budapest Treaty. The deposit will be irrevocably and without restriction or condition released to the public upon the issuance of a patent and for the enforceable life of the patent. Upon issuance of a patent, all restrictions upon the deposit will be removed, and the deposit is intended to meet the requirements of 37 CFR §§1.801-1.809. The deposit will be maintained in the depository for a period of 30 years, or 5 years after the last request, or for the effective life of the patent, whichever is longer, and will be replaced if necessary during that period.

### SEQUENCE INFORMATION

SEQ ID No: 1 Genetic SNP marker RF1. A genomic fragment of Apium graveolens L. dulce chromosome 8 is presented (positive strand), wherein the position of the SNP comprising a change from T to A is indicated as [T/A]. In this sequence, as well as in all other sequences listed herein, Y refers to any pyrimidine (C or T), M refers to A or C, R refers to any purine (A or G), W refers to A or T, K refers to G or T, and S refers to C or G.

ATATAGTGAAAAGAATAAGAGGGTGAGGATGGGGCTGTAGCCTGT
AGCACAAACGCACACAGGAGGTAGCTTAGCTCTGCTGGACCGCTT
CATGCCTCTC[T/A]TTTTTATGCCAAAAGTACAAACCAAAAAAT
AGTAAACAAAGCAATTGTATTTTCGAAAACATAAAACATTAGCCG
AAAGGAACTAAAACAATTGAGCATG

SEQ ID No: 2 Genetic SNP marker RF2.

ATGCATATCACGTAACACATAATCATGTTATTCATATATCACGTA
ATCACATATTCCATGTAACAYATAAGTCAGGTTGTCAAAAYATAG
GTTTTAGGAC[G/A]TTCAGAATTGAAATCGGGTCAATAACCGGG
TTTATCGATCAGCTAYCGACTCAGTATAACTCACAAATCAAATGA
CATTGCTATACAAAAGGAATTAGGT

SEQ ID No: 3 Forward primer that specifically recognizes the wildtype allele of the SNP in SEQ ID No: 1. Suitably it is labelled with FAM dye and used in a KASP™ marker assay in combination with SEQ ID No: 4 and SEQ ID No: 5.

GAAGGTGACCAAGTTCATGCTATTTTTTGGTTTGTACTTTTGGCA
TAAAAAA

SEQ ID No: 4 Forward primer that specifically recognizes the mutant allele of the SNP in SEQ ID No: 1. Suitably it is labelled with VIC dye and used in a KASP™ marker assay in combination with SEQ ID No: 3 and SEQ ID No: 5.

GAAGGTCGGAGTCAACGGATTCTATTTTTTGGTTTGTACTTTTGG
CATAAAAAT

SEQ ID No: 5 Common reverse primer (without a fluorophore label) that can be used in a KASP™ marker assay in combination with SEQ ID No: 3 and SEQ ID No: 4, to distinguish between the wildtype and mutant alleles of the SNP in SEQ ID No: 1.

TGCTGGACCGCTTCATGCCTCT

SEQ ID No: 6 Forward primer that specifically recognizes the wildtype allele of the SNP in SEQ ID No: 2. Suitably it is labelled with FAM dye and used in a KASP™ marker assay in combination with SEQ ID No: 7 and SEQ ID No: 8.

GAAGGTGACCAAGTTCATGCTGTTATTGACCCGATTTCAATTCTG
AAC

SEQ ID No: 7 Forward primer that specifically recognizes the mutant allele of the SNP in SEQ ID No: 2. Suitably it is labelled with VIC dye and used in a KASP™ marker assay in combination with SEQ ID No: 6 and SEQ ID No: 8.

GAAGGTCGGAGTCAACGGATTGGTTATTGACCCGATTTCAATTCT
GAAT

SEQ ID No: 8 Common reverse primer (without a fluorophore label) that can be used in a KASP™ marker assay in combination with SEQ ID No: 6 and SEQ ID No: 7, to distinguish between the wildtype and mutant alleles of the SNP in SEQ ID No: 2.

AGTCAGGTTGTCAAAAYATAGGTTTTAGGA

### BRIEF DESCRIPTION OF THE FIGURES/DRAWINGS

The patent or application file contains at least one drawing or figure executed in color. Copies of this patent or patent application publication with color drawing(s) will be provided by the Office upon request and payment of the necessary fec.

This disclosure may best be understood in conjunction with the accompanying figures, wherein:

FIG. 1 shows on the left side a typical wildtype celery leaf, with indication of the first internode (also known as the “stalk”), a second and third internode and a terminal leaflet. The dashed line indicates the usual position at which a celery leaf is cut during industrial processing, namely immediately below the first node. The part below the cutting level is commercially valuable, while the part above the cutting level is usually discarded as waste. On the right side, a typical leaf of a Rf-type celery plant of the invention is shown. It comprises two internodes on average, wherein the first internode is on average longer than in wildtype plants, and the second internode is always shorter than in wildtype plants. The overall result is that the proportion of leaf material that is discarded as waste is much lower than in wildtype celery plants.

FIG. 2 shows a comparison between representative wildtype and Rf-type celery plants that had been grown alongside each other in identical conditions and for the same amount of time. In panel A, intact celery plants from both types are compared. In panel B, individual leaves are shown from representative wildtype and Rf-type celery plants. In two cases the first internode is indicated with a bar (“1^{st}”), and arrows indicate the positions of nodes. The wildtype celery leaves in this figure have three internodes, whereas the Rf-type celery leaves have two internodes. It appears that the original first node is no longer present in the Rf-type and that the first internode thus comprises the original first and second internode.

FIG. 3 shows the average number of internodes per leaf in F1 progeny plants derived from three different crosses (Wt×Wt, Rf×Wt, and Rf×Rf).

FIG. 4 shows the average length (in millimeter) of the first and second leaf internodes, and the length of the total leaf, as measured in 15 wildtype (Wt) and in 15 Rf-type celery plants. One representative leaf was measured per plant.

The invention will be further illustrated in the Examples that follow, which are given for illustration purposes only and are not intended to limit the invention in any way.

### EXAMPLES

### Example 1

### Mapping of the Reduced Foliage Trait on Chromosome 8

In the research leading to the invention, plants with leaves of the Rf-type were observed.

A specific Rf-type celery plant (line 12.31110) was crossed (as a father) to a wildtype celery plant (Apium graveolens dulce) as a mother, and from this cross a segregating F2 progeny population of 282 plants was derived. In this population the Rf-type was observed to segregate in a monogenic recessive fashion, as determined by counting the total number of internodes per leaf petiole in the F2 progeny: of the 282 F2 plants, 77 had petioles with two internodes (=27.3%), 193 plants had petioles with three internodes, and 12 plants had petioles with four internodes. This corresponds to a three-to-one ratio of wildtype plants (with three or more internodes) versus Rf-type plants (with two internodes).

Based on this segregating population, a QTL mapping study was performed in which the genetic linkage of the Rf-phenotype was investigated to a set of molecular markers that were distributed evenly across all eleven chromosomes of the celery genome. In this study, a QTL region was identified on chromosome 8 that was closely linked to the presence of the Rf-type in celery plants. The QTL region was flanked by marker RFI (SEQ ID No. 1) and marker RF2 (SEQ ID No. 2). With reference to the public Api-gra_Ventura_v1 genome assembly (PMID 33095976; Song et al., 2020, Plant Biotechnol. 19:731-744), the QTL region of the invention was found to be located on chromosome 8 between positions 229,801,360 and 237,176,427. The genetic distance between the two flanking markers (RF1 and RF2) was observed to be about 0.1 cM.

Table 1 provides the sequences of the SEQ ID Nos. that can be used as markers, or that can be used to develop markers, to identify the presence of the QTL of the invention in a celery plant. Table 1 also lists the position of the SNP in each sequence, the derived (mutant) allele that is linked to the Rf trait, and the wildtype allele for each SNP, as well as the physical position of each SNP on the public Api-gra_Ventura_vl genome assembly.

To enable the unambiguous detection of the QTL region linked to the Rf-trait in celery breeding, and the selection of plants comprising said QTL region in their genome at any stage of their life cycle, primers were designed for the two flanking markers that were found to delimit said QTL region, for use in a KASP™ assay (KBiosciences). Table 2 provides a list of KASP primers that are suitable for the identification of the two flanking markers delimiting the QTL region.

### Example 2

### Morphological Characterization of the Reduced Foliage Trait in Celery

F1 progeny plants derived from three different crosses were grown in a field: Wt ×Wt (five plots with five plants per plot), Rf×Rf (seven plots with five plants per plot), and Rf×Wt (five plots with five plants per plot). For each plant, the total number of internodes was counted in five individual leaves, resulting in a total of 125 measurements for both the Wt×Wt and Rf×Wt crosses, and 175 measurements for the Rf×Rf cross.

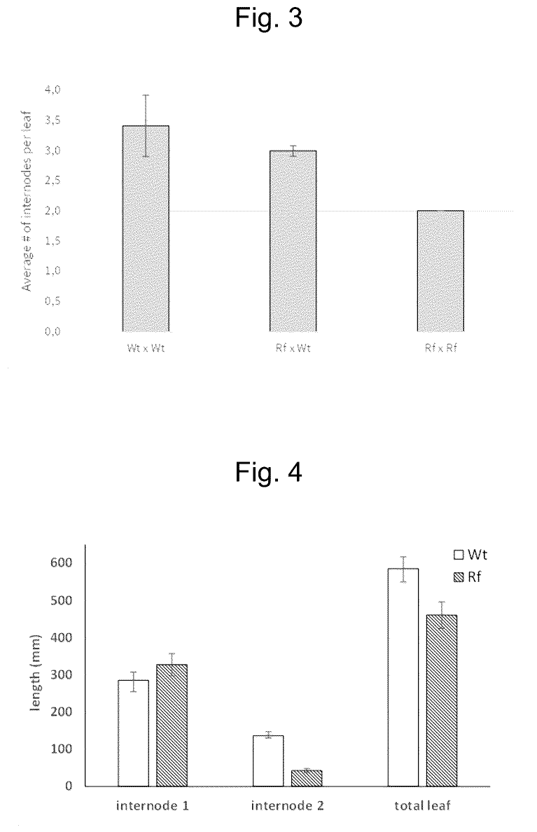

As shown in FIG. 3, we observed that celery plants derived from the Wt×Wt cross had on average 3.4+0.5 internodes per leaf, while the offspring of the Rf×Rf cross had on average 2.0+0.0 internodes per leaf. For the progeny derived from the Rf×Wt cross the average was 3.0+0.1 internodes per leaf. This experiment thus revealed that Rf-type celery plants that are homozygous for the Rf trait always have two internodes, whereas wildtype (Wt) celery plants usually have three or four internodes. Important to note is that the terminal leaflet has not been counted as an internode in this experiment.

In order to further describe and quantify the phenotype of Rf-type celery plants, 15 celery plants of the Rf-type were grown in a field alongside 15 wildtype celery plants. All leaves of the Rf-type plants had two internodes, and all leaves of the Wt plants had three internodes. The length of the first and second internodes of one representative leaf of each plant was measured, and the results are shown in FIG. 4. The first internode, which is the commercial product known as the celery stalk or stick, was observed to be on average slightly longer in Rf-type celery plants than in Wt celery plants: 328±30 mm in Rf-type plants compared to 285±23 mm in Wt plants. However, the second internode was significantly shorter in Rf-type celery plants than in Wt celery plants: 43+6 mm in Rf-type plants compared to 137±10 mm in Wt plants. The average total leaf length of the Wt celery plants was measured to be 586±32 mm, as compared to 461+36 mm for Rf-type plants. In Wt celery plants the first internode thus constituted about 50 percent of the total leaf length, whereas this ratio was about 71 percent in Rf-type celery plants.

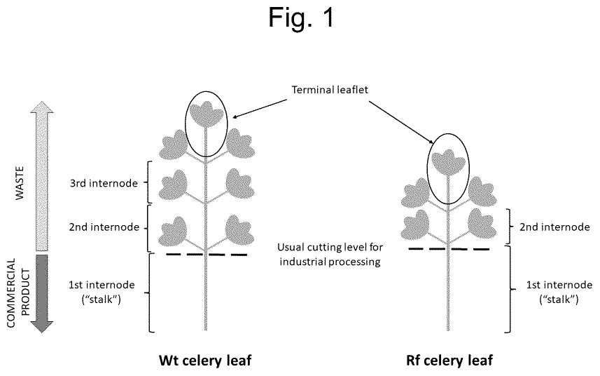

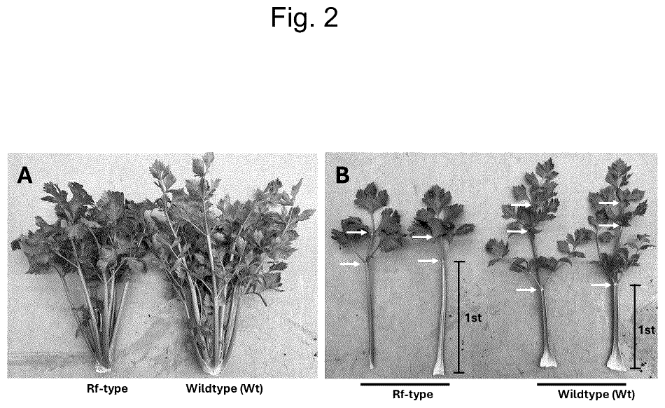

Taken together, the data from this experiment thus demonstrated that Rf-type celery plants have shorter leaves than Wt celery plants, but that they have a much higher commercially useful proportion than Wt celery plants, and that much less plant material needs to be discarded post-harvest. This is also illustrated in FIG. 1 and FIG. 2.

### Example 3

Introduction of the Reduced Foliage Trait into Other Celery Plants

Plants of the invention that were deposited under NCIMB accession number 44381 were crossed with wildtype celery plants that did not display the Rf phenotype. The F2 progeny segregated for plants that showed the same characteristics as the parent plant of NCIMB accession number 44381, more specifically having the Rf trait of the invention. The segregation was in a monogenic recessive fashion. These plants could be identified and selected from among the F2 progeny population by using the KASP primers developed in Example 1 and presented in Table 2. Further development of these plants resulted in lines and hybrid varieties with the Rf trait of the invention, as found in NCIMB accession number 44381.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A cultivated Apium graveolens L. dulce plant comprising a QTL on chromosome 8 which is located between SEQ ID No. 1 and SEQ ID No. 2, which QTL when homozygously present is responsible for the presence of two internodes on average per leaf at the harvesting stage.

2. A cultivated Apium graveolens L. dulce plant as claimed in claim 1, wherein the average number of internodes per leaf at the harvesting stage is two.

3. A cultivated Apium graveolens L. dulce plant as claimed in claim 1 or 2, wherein the QTL on chromosome 8 is genetically linked to at least one of the SNPs as presented in SEQ ID No. 1 and SEQ ID No. 2.

4. A cultivated Apium graveolens L. dulce plant as claimed in claim 1, wherein the QTL is as comprised in the genome of an Apium graveolens L. dulce plant representative seed of which was deposited with the NCIMB under deposit number NCIMB 44381 or NCIMB 41513.

5. A cultivated Apium graveolens L. dulce plant as claimed in claim 1, wherein the QTL is introgressed from a plant grown from seed deposited with the NCIMB under NCIMB 44381 or NCIMB 41513, or from a progeny plant thereof that has retained the QTL.

6. An Apium graveolens L. dulce seed of, from, or that produces the cultivated Apium graveolens L. dulce plant as claimed in claim 1 and comprises the QTL on chromosome 8.

7. An Apium graveolens L. dulce seed comprising a QTL on chromosome 8 which is located between SEQ ID No. 1 and SEQ ID No. 2, which QTL when homozygously present is responsible for the presence of two internodes on average per leaf at the harvesting stage of a cultivated Apium graveolens L. dulce plant grown from the seed.

8. Propagation material from, or capable of developing into and/or derived from an Apium graveolens L. dulce plant according to claim 1, wherein the propagation material is suitable for sexual reproduction, and comprises a microspore, pollen, an ovary, an ovule, an embryo sac, or an egg cell; or wherein the propagation material is suitable for vegetative reproduction, and comprises a cutting, a root, a stem, a cell, or a protoplast; or wherein the propagation material is suitable for tissue culture of regenerable cells, and comprises a leaf, pollen, an embryo, a cotyledon, a hypocotyl, a meristematic cell, a root, a root tip, an anther, a flower, a seed, or a stem; and wherein the plant produced from the propagation material comprises the QTL on chromosome 8.

9. A method for producing a cultivated Apium graveolens L. dulce plant having two internodes on average per leaf at the harvesting stage, said method comprising:
a) crossing a first Apium graveolens L. dulce plant with a second Apium graveolens L. dulce plant to obtain a first generation population, wherein the first Apium graveolens L. dulce plant is an Apium graveolens L. dulce plant according to claim 1;
b) performing one or more rounds of selfing and/or crossing of the plant resulting from the cross to obtain a further generation population;
c) phenotypically selecting from among the plants resulting from the further generation population of step b) a plant that has two internodes on average per leaf at the harvesting stage.

10. The method as claimed in claim 9, wherein the first plant is a plant grown from seed deposited under NCIMB accession number NCIMB 44381 or NCIMB 41513, or a progeny plant thereof that has retained the QTL on chromosome 8.

11. A method for producing Apium graveolens L. dulce seed comprising the QTL on chromosome 8 comprising producing a cultivated Apium graveolens L. dulce plant according to claim 9, and harvesting seed therefrom.

12. A method for producing Apium graveolens L. dulce seed comprising the QTL on chromosome 8 comprising producing a cultivated Apium graveolens L. dulce plant according to claim 10, and harvesting seed therefrom.

13. A method for producing cultivated hybrid Apium graveolens L. dulce seed comprising crossing a first Apium graveolens L. dulce parent plant with a second Apium graveolens L. dulce parent plant and harvesting the resultant hybrid seed, wherein the first parent plant and the second parent plant are plants comprising homozygously a QTL on chromosome 8 which is located between SEQ ID No. 1 and SEQ ID No. 2, which QTL when homozygously present in an Apium graveolens L. dulce plant is responsible for the presence of two internodes on average per leaf at the harvesting stage.

14. The cultivated hybrid Apium graveolens L. dulce seed produced by the method of claim 11.

15. The cultivated hybrid Apium graveolens L. dulce seed produced by the method of claim 12.

16. The cultivated hybrid Apium graveolens L. dulce seed produced by the method of claim 13.

17. A method for growing a cultivated Apium graveolens L. dulce plant with two internodes on average per leaf at the harvesting stage, which method comprises the step of germinating a seed according to claim 14.

18. A method for growing a cultivated Apium graveolens L. dulce plant with two internodes on average per leaf at the harvesting stage, which method comprises the step of germinating a seed according to claim 15.

19. A method for growing a cultivated Apium graveolens L. dulce plant with two internodes on average per leaf at the harvesting stage, which method comprises the step of germinating a seed according to claim 16.
